(19) United States
(12) Patent Application Publication
(10) Pub. No.: US 2022/0233144 A1
(43) Pub. Date: Jul. 28, 2022

(54) APPARATUS AND METHODS FOR SECURING SENSORS

(71) Applicant: TT1 Products, Inc., Atlanta, GA (US)

(72) Inventors: Todd Furneaux, Atlanta, GA (US); Harold Philpott Southerland, III, Atlanta, GA (US)

(21) Appl. No.: 17/155,578

(22) Filed: Jan. 22, 2021

(51) Int. Cl. A61B 5/00

(52) U.S. Cl. CPC A61B 5/6832

(57) ABSTRACT

In various embodiments, a device may include a sensor cover and a disk-shaped portion, such that the sensor cover holds a sensor securely and comfortably to a user's body. In at least one embodiment, the sensor cover may be made of lightweight, durable materials such as silicon. In one or more embodiments, the disk-shaped portion may include an adhesive substance applied to one side of the disk-shaped portion. In several embodiments, the sensor cover includes channels that allow for moisture and air inside the sensor cover to escape, which decreases skin irritation and protects against the generation of an unclean environment inside the sensor cover. In at least one embodiment, the channels create airways between the inside of the sensor cover and the outside environment, thus creating pathways for air and moisture to exit the inside of the sensor cover.

### BACKGROUND

Biosensors are devices used to detect the presence or concentration of a substance in a human body. Some biosensors may have a portion of the biosensor implanted into a user to allow for continuous substance monitoring. For example, an implanted portion may include a filament inserted into the user's skin. These biosensors utilize bioreaders and electronic communication devices, located on the outside the user's body, to read substance information from the implanted portion of the device and to send information regarding the amount of substance in the user's body to the user (e.g., to the user's mobile device). Because the parts of the biosensor are located outside the user's body, the biosensor may easily be dislodged, disconnected, and removed from the user's body.

Therefore, there is a long-felt but unresolved need for an apparatus that securely and comfortably attaches a biosensor to a user.

### BRIEF SUMMARY OF THE DISCLOSURE

Briefly described, and according to one embodiment, aspects of the present disclosure generally relate to a device/apparatus and methods for attaching a biosensor to a user. In various embodiments, a device may include a sensor cover and a disk-shaped portion, such that the sensor cover holds a sensor securely and comfortably to a user's body. In at least one embodiment, the sensor cover may be made of lightweight, durable materials such as silicon. In one or more embodiments, the disk-shaped portion may include an adhesive substance applied to one side of the disk-shaped portion that is generally safe for a user's body (e.g., not a known irritant).

In several embodiments, the sensor cover includes channels that allow for moisture and air inside the sensor cover to escape, which further decreases skin irritation and protects against the generation of an unclean environment inside the sensor cover. In at least one embodiment, the channels create airways between the inside of the sensor cover and the outside environment, thus creating pathways for air and moisture to exit the inside of the sensor cover.

In multiple embodiments, the present device may include one or more adhesive covers for covering the adhesive prior to a user removing the adhesive cover and using the device to secure a sensor to the user. In some embodiments, the adhesive covers may include tabs to simplify the removal of the adhesive covers from the adhesive.

According to a first aspect, an apparatus for securing a biosensor to a human comprising: a sensor cover for receiving a biosensor device, the cover comprising: a generally conical-shaped body defining a top opening and a bottom opening and comprising: an exterior surface sloping from the top opening to the bottom opening; an interior surface between the top opening and bottom opening comprising one or more protruding portions around a perimeter of the top opening; one or more channels extending from the interior surface into the body; and a rim extending outwardly from the exterior surface comprising an upper surface substantially perpendicular to a portion of the sloping exterior surface; a disk defining a disk opening and comprising a bottom surface; and an adhesive applied to the disk bottom surface for securing the disk to the sensor cover.

According to a second aspect, the apparatus of the first aspect or any other aspect, wherein the one or more protruding portions define a least a portion of the one or more channels.

According to a third aspect, the apparatus of the second aspect or any other aspect, wherein the one or more channels extend from the perimeter of the top opening to a first point on the interior surface.

According to a fourth aspect, the apparatus of the third aspect or any other aspect, wherein the one or more protruding portions extend from the perimeter of the top opening to a second point on the interior surface.

According to a fifth aspect, the apparatus of the fourth aspect or any other aspect, wherein the first point is further from the perimeter of the top opening then the second point.

According to a sixth aspect, the apparatus of the fifth aspect or any other aspect, wherein each of the one or more channels extend from the perimeter of the top opening approximately 3 millimeters to the first point.

According to a seventh aspect, the apparatus of the sixth aspect or any other aspect, wherein the disk opening comprises a diameter greater than a diameter of the body bottom opening.

According to an eighth aspect, the apparatus of the seventh aspect or any other aspect, wherein the disk is secured to the sensor cover by the adhesive attaching to the rim of sensor cover.

According to a ninth aspect, the apparatus of the eighth aspect or any other aspect, wherein the disk and sensor cover are secured to a human via a portion of the adhesive not attached to the rim of the sensor cover.

According to a tenth aspect, the apparatus of the ninth aspect or any other aspect, wherein the apparatus further comprises one or more adhesive covers removably attached to the adhesive to protect the adhesive prior to securing the disk to the sensor cover.

According to an eleventh aspect, an apparatus for securing a biosensor to a human comprising: a sensor cover for receiving a biosensor device, the cover comprising: a generally conical-shaped body defining a top opening and a bottom opening and comprising: an exterior surface sloping from the top opening to the bottom opening; an interior surface between the top opening and bottom opening; one or more channels extending from the interior surface into the body; and a rim extending outwardly from the exterior surface comprising an upper surface substantially perpendicular to a portion of the sloping exterior surface; a disk defining a disk opening and comprising a bottom surface; and an adhesive applied to the disk bottom surface for securing the disk to the sensor cover.

According to a twelfth aspect, the apparatus of the eleventh aspect or any other aspect, wherein the interior surface further comprises one or more protruding portions around a perimeter of the top opening.

According to a thirteenth aspect, the apparatus of the twelfth aspect or any other aspect, wherein the one or more protruding portions define a least a portion of the one or more channels.

According to a fourteenth aspect, the apparatus of the thirteenth aspect or any other aspect, wherein the one or more channels extend from the perimeter of the top opening to a first point on the interior surface.

According to a fifteenth aspect, the apparatus of the fourteenth aspect or any other aspect, wherein the one or more protruding portions extend from the perimeter of the top opening to a second point on the interior surface.

According to a sixteenth aspect, the apparatus of the fifteenth aspect or any other aspect, wherein the first point is further from the perimeter of the top opening then the second point.

According to a seventeenth aspect, the apparatus of the sixteenth aspect or any other aspect, wherein each of the one or more channels extend from the perimeter of the top opening approximately 3 millimeters to the first point.

According to an eighteenth aspect, the apparatus of the sixteenth aspect or any other aspect, wherein the disk opening comprises a diameter greater than a diameter of the body bottom opening.

According to a nineteenth aspect, the apparatus of the eighteenth aspect or any other aspect, wherein the disk is secured to the sensor cover by the adhesive attaching to the rim of sensor cover.

According to a twentieth aspect, the apparatus of the nineteenth aspect or any other aspect, wherein the disk and sensor cover are secured to a human via a portion of the adhesive not attached to the rim of the sensor cover.

According to a twenty-first aspect, the apparatus of the twentieth aspect or any other aspect, wherein the apparatus further comprises one or more adhesive covers removably attached to the adhesive to protect the adhesive prior to securing the disk to the sensor cover.

These and other aspects, features, and benefits of the claimed invention(s) will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE FIGURES

The accompanying drawings illustrate one or more embodiments and/or aspects of the disclosure and, together with the written description, serve to explain the principles of the disclosure. Wherever possible, the same reference numbers are used throughout the drawings to refer to the same or like elements of an embodiment, and wherein:

FIG. 1 is a top perspective view of an exemplary device, according to one embodiment of the present application;

FIG. 2 is a bottom perspective view of an exemplary device, according to one embodiment of the present application;

FIG. 3 is a top view of an exemplary device, according to one embodiment of the present application;

FIG. 4 is a bottom view of an exemplary device, according to one embodiment of the present application;

FIG. 5 is a side view of an exemplary device, according to one embodiment of the present application;

FIG. 6 is an exploded view of an exemplary device, according to one embodiment of the present application;

FIG. 7 is a bottom view of an exemplary device, according to one embodiment of the present application;

FIG. 8 is a top perspective view of an exemplary device, according to one embodiment of the present application.

### DETAILED DESCRIPTION

For the purpose of promoting an understanding of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined in accordance with and as expressed in the claims.

Whether a term is capitalized is not considered definitive or limiting of the meaning of a term. As used in this document, a capitalized term shall have the same meaning as an uncapitalized term, unless the context of the usage specifically indicates that a more restrictive meaning for the capitalized term is intended. However, the capitalization or lack thereof within the remainder of this document is not intended to be necessarily limiting unless the context clearly indicates that such limitation is intended.

### Overview

In various embodiments, the overpatch device may comfortably secure a biosensor to a user. In one or more embodiments, the overpatch device includes a disk-shaped portion and a sensor cover. In at least one embodiment, the sensor cover may be placed over and around the biosensor, such an interior of the sensor cover creates a space for the biosensor to fit securely (and against the user's body).

In several embodiments, the overpatch device may include a disk-shaped portion and a sensor cover. In some embodiments, the disk-shaped portion has a top and bottom surface. In various embodiments, the bottom surface may include an adhesive that allows a user to adhere the overpatch device onto the body of a user. In at least one embodiment, the disk-shaped portion may be in part made from a material that allows a user to stretch the disk-shaped portion to create a tight fit over the sensor cover and/or biosensor.

In one or more embodiments, the sensor cover may be generally conical-shaped and fit over a biosensor. In some embodiments, the sensor cover may define a circular opening at the top of the sensor cover so that a user may be able to see the biosensor when the overpatch device is placed over the biosensor.

In at least one embodiment, the sensor cover may include one or more channels at least partially formed or defined by one or more blocks. In some embodiments, the one or more blocks extend from the bottom surface of the sensor cover with spaces in between each block. In many embodiments, the spaces between adjacent blocks of the one or more blocks form a channel (which may extend into the bottom surface of the sensor cover). In one embodiment, the one or more blocks may be any suitable shape.

### Exemplary Embodiments

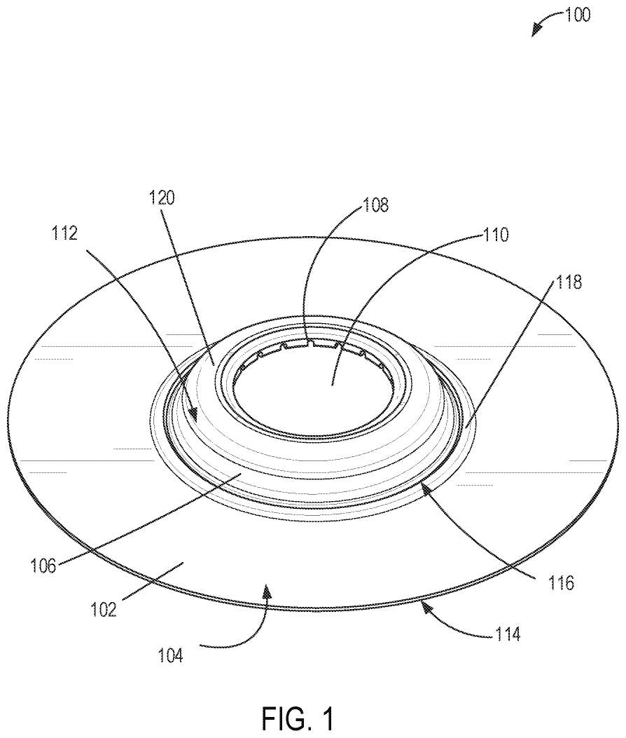

Referring now to the figures, for the purposes of example and explanation of the fundamental processes and components of the disclosed systems and methods, reference is made to FIG. 1, which illustrates an exemplary, high-level overview 100 of one embodiment of an overpatch device. As will be understood and appreciated, the exemplary, high-level overview 100 shown in FIG. 1 represents merely one approach or embodiment of the present device, and other aspects are used according to various embodiments of the present device.

As shown in FIG. 1, a top perspective view of an exemplary overpatch device 100 is shown, according to one embodiment of the present disclosure. In various embodiments, the overpatch device 100 includes a disk-shaped portion 102 and a sensor cover 106 and may operate to cover and attach a biosensor to a human.

In multiple embodiments, the overpatch device 100 may include a disk-shaped portion 102. In one or more embodiments, the disk-shaped portion 102 may be disk or ring shaped, such that the disk-shaped portion 102 has an outer edge or perimeter 114 in the shape of a circle. In some embodiments, the disk-shaped portion 102 may be rectangular, oval, or any other suitable shape.

In some embodiments, the disk-shaped portion 102 may have a first diameter based on the circumference of the outer edge 114. In many embodiments, the first diameter based on the circumference of the outer edge 114 may be any suitable diameter. In one embodiment, the first diameter based on the circumference of the outer edge 114 may be approximately 76.0 millimeters. In at least one embodiment the first diameter may be approximately 70-80 millimeters. In several embodiments, the first diameter may be approximately 67.70 to about 76.30 millimeters.

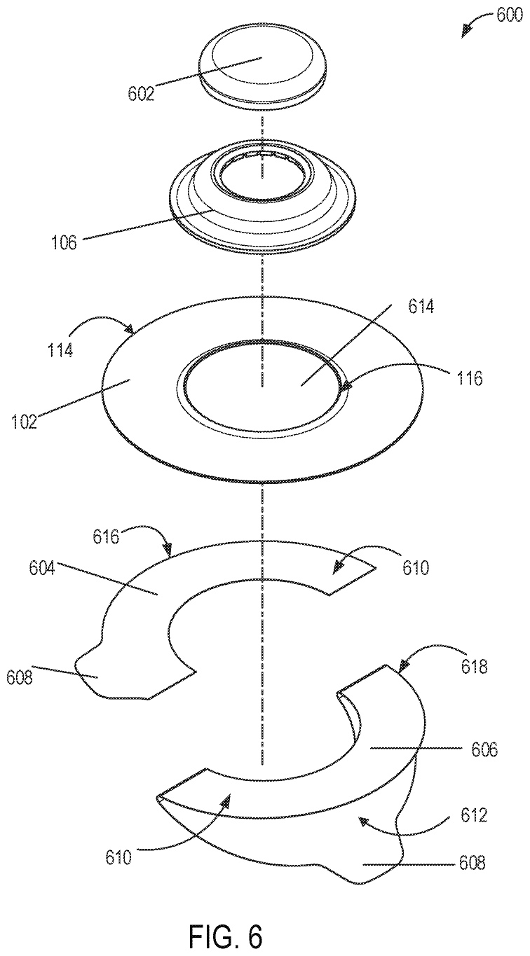

In at least one embodiment, the disk-shaped portion 102 may define a circular opening 614 (see FIG. 6) that has an inner edge or perimeter 116. In many embodiments, the inner edge 116 may have a second diameter that is smaller than the first diameter (defined by the outer edge 114). In some embodiments, the second diameter based on the circumference of the inner edge 116 may be any suitable diameter that is less than the first diameter defined by the outer edge 114.

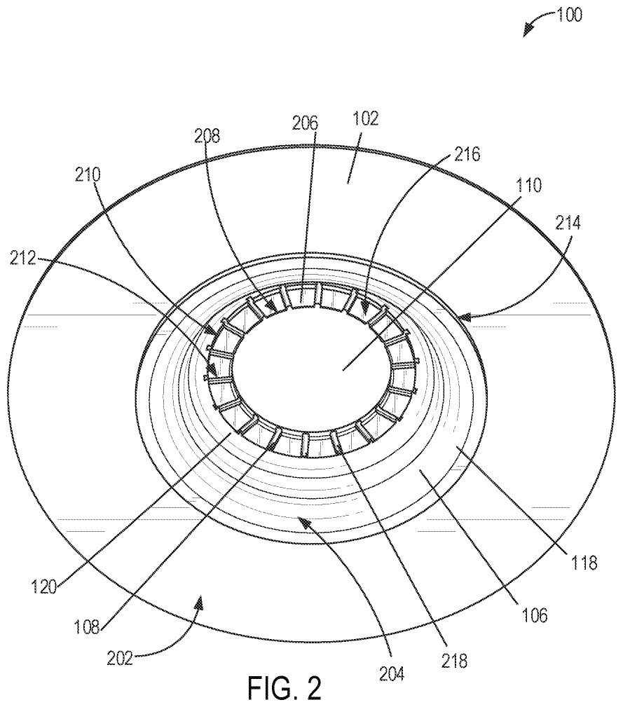

In some embodiments, the disk-shaped portion 102 may have a top surface 104 and a bottom surface 202 (see FIG. 2). In many embodiments, the disk-shaped portion 102 may include a certain thickness between the top surface 104 and the bottom surface 202. In at least one embodiment, the thickness between the top surface 104 and the bottom surface 202 may be approximately 0.60 millimeters. In at least one embodiment, the thickness between the top surface 104 and the bottom surface 202 may be approximately 0.550 to about 0.650 millimeters. In one or more embodiments, the thickness between the top surface 104 and the bottom surface 202 may be approximately 0.10 to 1.0 millimeters. In one embodiment, the top surface 104 may be between the outer edge 114 and the inner edge 116, and the interior surface 204 (see FIG. 2) may be between the outer edge 114 and the inner edge 116 on the opposing side of the disk-shaped portion 102.

In multiple embodiments, the circular opening 614 (see FIG. 6) defined by the inner edge 116 may be in the middle of the disk-shaped portion 102 such that the distance between the outer edge 114 and the inner edge 116 of the disk-shaped portion 102 is approximately constant (e.g., the width of top surface 104 and interior surface 204 is approximately constant around the disk-shaped portion). In several embodiments, the distance between the outer edge 114 and the inner edge 116 of the disk-shaped portion 102 may be approximately 21.0 millimeters. In at least one embodiment, the distance between the outer edge 114 and the inner edge 116 may be approximately 15.0 to about 22.0 millimeters. In many embodiments, the distance between the outer edge 114 and the inner edge 116 may be approximately 20.0 to about 30.0 millimeters. In some embodiments, the circular opening defined by the inner edge 116 may not be in the middle of the disk-shaped portion 102. In further embodiments, the inner edge 116 may define an opening that is not circular, but is an oval shape, a quadrilateral shape, triangular, or any other suitable shape.

The disk-shaped portion 102 may be constructed from any suitable material. For example, in one embodiment, the disk-shaped portion 102 may be constructed from plastic (e.g., PVC, polyethylene, polyurethane, etc.), woven fabric, latex, and/or foam. In at least one embodiment, the top surface 104 or bottom surface 202 may include a surface material (e.g., a gloss) that covers the disk-shaped portion 102 material (e.g., plastic, as discussed above).

As further discussed herein, in several embodiments, the bottom surface 202 may be coated with an adhesive material such that the bottom surface 202 may adhere to a user and/or to at least a portion of the sensor cover 106. In one or more embodiments, the adhesive material may be an acrylate/acrylic, silicon, rubber, or other pressure-sensitive adhesives.

In at least one embodiment, the disk-shaped portion 102 may include an elastic material that allows a user to stretch the disk-shaped portion 102 to ensure a tight bond on the user. For example, in one embodiment, the disk-shaped portion 102 may be constructed of or include different types of tape (e.g., the tape adhesive is the adhesive material on the bottom surface 202) suitable for adhering to a user.

In various embodiments, the overpatch device 102 may include a sensor cover 106. In one or more embodiments, the sensor cover 106 may be placed over a biosensor and attached to a user, so that the biosensor fits underneath the sensor cover 106.

In multiple embodiments, the sensor cover 106 may be constructed from any suitable material. For example, in one embodiment, the sensor cover 106 may be constructed from a lightweight material, such as silicon, plastic, and/or other similar materials. In at least one embodiment, the sensor cover 106 may be constructed via injection molding, 3D printing, or other similar production methods.

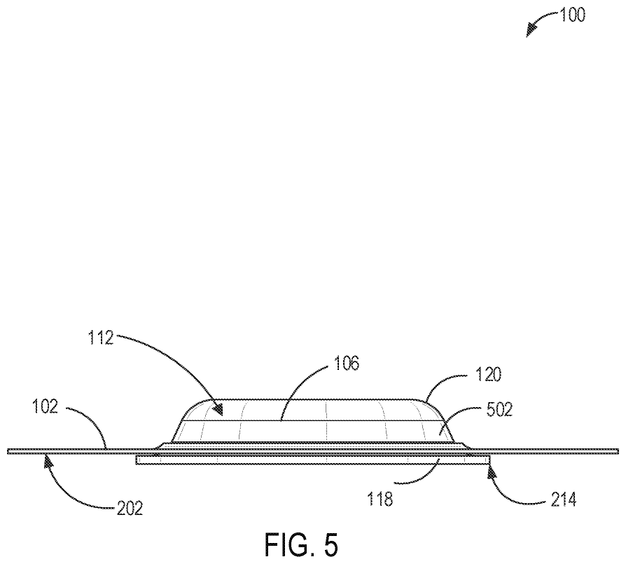

In several embodiments, the sensor cover 106 may be conical-shaped and include a base (also referred to herein as a “rim”) 118, a top area 120, and a sloped middle area 502 (see FIG. 5) that connects the base 118 to the top area 120. In at least one embodiment, the base 118 may be the bottom portion of the sensor cover 106 and have an outer edge 214 (see FIG. 2) that is circular and has a particular diameter. In some embodiments, the outer edge 214 of the base 118 may have a larger diameter than the biosensor.

In various embodiments, the top area 120 may define a circular opening 110 having a particular diameter. In one embodiment, the diameter of the outer edge 214 of the base 118 may be larger than the diameter of the circular opening 110 as defined by the top area 120, such that the sensor cover 106 slopes downwardly from the top area 120 toward the base 118. In at least one embodiment, the diameter of the circular opening 110 as defined by the top area 120 may be approximately 20.0 millimeters. In one or more embodiments, the diameter of the circular opening 110 may be approximately 19.80 to about 20.20 millimeters. In many embodiments, the diameter of the circular opening 110 may be approximately 0.10 to about 20.0 millimeters. In some embodiments, the diameter of the circular opening 110 may be approximately 20.0 to 30.0 millimeters. In an alternative embodiment, the top area 120 may not define a circular opening 110.

In multiple embodiments, the sensor cover 106 may include an exterior surface 112 and an interior surface 204 (see FIG. 2). In some embodiments, the exterior surface 112 and the interior surface 204 may be generally smooth and, in at least one embodiment, may include a gloss or other surface agent included on the sensor cover 106. In one or more embodiments, the sensor cover 106 may have a certain thickness between the exterior surface 112 and the interior surface 204. In at least one embodiment, the thickness between the exterior surface 112 and the interior surface 204 may be any suitable thickness for covering and securing the sensor to the user. In one embodiment, the thickness between the exterior surface 112 and the interior surface 204 is approximately 1.53 millimeters. In many embodiments, the thickness between the exterior surface 112 and the interior surface 204 is approximately 1.45 to about 1.60 millimeters. In one embodiment, the thickness between the exterior surface 112 and the interior surface 204 is approximately 0.40 to about 1.45 millimeters.

In various embodiments, the sensor cover 106 may include one or more channels 108. In at least one embodiment, the one or more channels 108 are integrally formed with and defined by the interior surface 204 of the sensor cover 106 and intersect with the opening 110 (e.g., the one or more channels 108 may be located on the edge of the exterior surface 112). In one or more embodiments, the one or more channels 108 create a gap in the interior surface 204 of the sensor cover 106 so that air and moisture may flow from inside the overpatch device 100 to the outside environment via the opening 110 to decrease skin irritation and other hygienic issues (e.g., mildew, etc.).

In many embodiments, the disk-shaped portion 102 may be operatively connected to the sensor cover 106. In at least one embodiment, the disk-shaped portion 102 may be attached to the sensor cover 106 by adhering the adhesive bottom surface 202 of the disk-shaped portion 102 onto the exterior surface 112 of the sensor cover 106 proximate to the base 118. In one embodiment, because the disk-shaped portion 102 may be adhered to the sensor cover 106, a portion of the base 118 may be under the disk-shaped portion 102 (see FIG. 5). In another embodiment, the disk-shaped portion 102 and the sensor cover 106 may be a single unit created via injection molding or a similar process, such that the disk-shaped portion 102 is an extension of the base 118 and made of the same material as the sensor cover 106. In yet another embodiment, the disk-shaped portion 102 may have an adhesive on the top surface 104 around the inner edge 116, and the interior surface 204 of the base 118 of the sensor cover 106 may be placed on top of the adhesive on the top surface 104 of the disk-shaped portion 102 so that the sensor cover 106 adheres to the adhesive on the top surface of the disk-shaped portion 102. In yet another environment, the sensor cover 106 and disk-shaped portion 102 may connect via a hook-and-loop fastener, snap-fits, threading, or other similar connection methods.

Turning now to FIG. 2, a bottom perspective view of an exemplary overpatch device 100 is shown, according to one embodiment of the present disclosure. In one or more embodiments, and as described above, the disk-shaped portion 102 may include the bottom surface 202, and the sensor cover 106 may include the interior surface 204. In various embodiments, the sensor cover 106 may slope up and inwards from the outer edge 214 of the base 118 to the top area 120 of the sensor cover 106.

In various embodiments, the outer edge 214 of the base 118 may have a particular diameter. In one or more embodiments, the diameter of the outer edge 214 as defined by the base 118 may be approximately 44.0 millimeters. In one or more embodiments, the diameter of the outer edge 214 may be approximately 43.80 to about 44.20 millimeters. In one embodiment, the diameter of the outer edge 214 may be approximately 40.0-50.0 millimeters. In some embodiments, the outer edge 214 of the base 118 may have a larger diameter than the biosensor.

In multiple embodiments, sensor cover 106 may include the one or more channels 108 at the top area 120. In one or more embodiments, the one or more channels 108 may intersect with the opening 110 on the interior surface 204 of the sensor cover 106, such that the air and/or moisture may vent through the channels into the outside environment.

In at least one embodiment, the one or more channels 108 may be at least partially defined by one or more blocks 206 integrally formed with the interior surface 204 of the sensor cover 106 (e.g., interior surface 204 transitions into the one or more blocks 206) that intersects the opening 110. In some embodiments, the one or more blocks 206 may be formed at particular intervals such that the one or more channels 108 between the one or more blocks 206 have a certain width. In several embodiments, the one or more blocks 206 and sensor cover 106 may be formed together via injection molding or other similar processes. In at least one embodiment, the one or more blocks 206 may connect to the sensor cover 106 via an adhesive, a hook and loop fastener, threading, snap-fits, or other similar connection methods.

As shown in FIG. 2, the one or more blocks 206 may be a particular shape. In several embodiments, the one or more blocks 206 may be three-dimensional shapes. In multiple embodiments, each of the one or more blocks 206 may be the same shape, or may be different shapes. For example, in one embodiment, the one or more blocks 206 may be a quadrilateral-shaped prism (as seen in FIG. 2). In at least one embodiment, the one or more blocks 206 may also be shaped as a cylinder, a cube, a cone, a pyramid, a triangular prism, a rectangular prism, a cuboid, a sphere (or a portion of a sphere, e.g., a hemisphere) and/or a polyhedron.

In various embodiments, each of the one or more blocks 206 may include a front edge 208 that intersects with the opening 110. In some embodiments, each of the one or more blocks 206 may also include a back edge 210 and one or more side edges 212, depending on the particular shape of each of the one or more blocks 206. In at least one embodiment, the front edge 208 of each of the one or more blocks 206 may be approximately 2.68 millimeters in length. In many embodiments, the front edge 208 of each of the one or more blocks 206 may be approximately 2.50 to about 3.00 millimeters in length. In one or more embodiments, the front edge 208 of each of the one or more blocks 206 may be approximately 3.0 to about 4.0 millimeters in length. In one embodiment, the front edge 208 of each of the one or more blocks 206 may be approximately 4.0 to about 6.0 millimeters in length. In some embodiments, the front edge 208 of each of the one or more blocks 206 may be approximately 1.0 to about 2.5 millimeters.

In multiple embodiments, the back edge 210 of each of the one or more blocks 206 may be approximately 3.72 millimeters in length. In at least one embodiment, the back edge 210 of each of the one or more blocks 206 may be approximately 3.50 to about 4.0 millimeters in length. In many embodiments, the back edge 210 of each of the one or more blocks 206 may be approximately 4.0 to about 8.0 millimeters in length. In one embodiment, the back edge 210 of each of the one or more blocks 206 may be approximately 1.0 to about 3.50 millimeters in length.

In one or more embodiments, the each of the one or more side edges 212 of the one or more blocks 206 may be approximately 3.0 millimeters in length. In some embodiments, each of the one or more side edges 212 may be approximately 2.5 to about 3.5 millimeters in length. In at least one embodiment, each of the one or more side edges 212 may be approximately 3.5 to about 5.0 millimeters in length. In one embodiment, each of the one or more side edges 212 may be approximately 0.5 to about 2.5 millimeters in length.

In many embodiments, the front edge 208 and the back edge 210 may be straight edges, or may reflect the shape of the opening 110. For example, in one embodiment, the opening 110 may be generally circular (or an oval) and the one or more blocks may be approximately quadrilateral prisms, so the front edge 208 and the back edge 210 of each of the one or more blocks 206 may have the same arc angle as the opening 110 (thus, in this example, the back edge 210 is longer than the front edge 208). In another example, in some embodiments, the opening 110 may be a square, and the one or more blocks may be a rectangular prism, so the front edge 208 and the back edge 210 of each of the one or more blocks 206 may be straight and equal in length. In at least one embodiment, the one or more blocks 206 may also be shaped as a cylinder, a cube, a cone, a pyramid, a triangular prism, a rectangular prism, a cuboid, a sphere (or a portion of a sphere, e.g., a hemisphere) and/or a polyhedron. In one or more embodiments, depending on the shape of the one or more blocks 206, the front edge 208 may not reflect the shape of the opening 110 (e.g., if the one or more blocks 206 are cylindrical, the front edge 208 is the only edge and may intersect the opening 110 but not reflect the shape of the opening 110).

In various embodiments, the front edge 208, the back edge 210, and/or the one or more side edges 212 of each of the one or more blocks 206 may define a block surface 216 for each of the one or more blocks 206. In at least one embodiment, the block surface 216 of each of the one or more blocks 206 may be generally parallel to the interior surface 204 of the sensor cover 106.

In multiple embodiments, the one or more blocks 206 may extend or protrude from the interior surface 204 of the sensor cover 106, such that each of the one or more blocks 206 have a certain height. In at least one embodiment, the height of the one or more blocks 206 may be defined as the distance between the interior surface 204 of the sensor cover 106 and the block surface 216. In many embodiments, each of the one or more blocks 206 may have a height of approximately 1.0 millimeters. In one or more embodiments, each of the one or more blocks 206 may have a height of approximately 0.1 to about 1.0 millimeters. In one embodiment, each of the one or more blocks 206 may have a height of approximately 0.75 to about 1.50 millimeters. In several embodiments, each of the one or more blocks 206 may have a height of approximately 1.5 to about 5.0 millimeters. In some embodiments, each of the one or more blocks 206 may extend a different height from the sensor cover 106, or each of the one or more blocks 206 may extend the same height from the sensor cover 106.

In many embodiments, the one or more blocks 206 may be integrally formed or otherwise connected to the sensor cover 106 such that there is a space in between each of the one or more blocks 206 that define the one or more channels 108. In one or more embodiments, the one or more blocks 206 may be integrally formed such that the one or more blocks 206 are a particular distance from each other. In another embodiment, the one or more blocks 206 may be formed with different distances between each of the blocks. For example, in one embodiment, a particular block may be one millimeter away from a second block adjacent to the particular block, and may be two millimeters away from a third block that is adjacent to the particular block on the opposing side of the particular block.

In various embodiments, each of the one or more channels 108 may have a similar length as the one or more side edges 212 of the one or more blocks 206. In one embodiment, the one or more channels 108 may extend further than one or more side edges 212 of the one or more blocks 206 (as shown in FIG. 2). As shown in FIG. 2, the one or more channels 108 extends past the back edge 210 of the one more blocks 206.

In several embodiments, each of the one or more channels 108 may include a channel floor 218. In one or more embodiments, each channel floor 218 of the one or more channels 108 may be the interior surface 204 of the sensor cover 106 in the spaces between the one or more blocks 206 that define the one or more channels 108. In some embodiments, each channel floor 218 may be a flat surface such that the distance between the channel floor 218 and the block surface 216 is constant across the flat surface of the channel floor. In at least one embodiment, each channel floor 218 may have a curved surface such that the distance from the channel floor 218 to the block surface 216 varies across the curved surface of the channel floor 218.

In multiple embodiments, the one or more channels 108 are at least partially defined by the exterior surface 112 of the sensor cover 106 so that portion of the sensor cover 106 that contains a channel of the one or more channels 108 has a smaller thickness than a portion of the sensor cover 106 that does not contain a channel of the one or more channels 106. In at least one embodiment, the thickness of a portion of the sensor cover 106 that contains a channel of the one or more channels 108 may be the distance from the exterior surface 112 of the sensor cover 106 to the channel floor 218 of the channel. In one embodiment, the thickness of the portion of the sensor cover 106 that contains a channel of the one or more channels 108 may be approximately 0.46 millimeters. In many embodiments, the thickness of the portion of the sensor cover 106 that contains a channel of the one or more channels 108 may be approximately 0.40 to about 0.50 millimeters. In one or more embodiments, the thickness of the portion of the sensor cover 106 that contains a channel of the one or more channels 108 may be approximately 0.10 to about 0.40 millimeters. In several embodiments, the thickness of the portion of the sensor cover 106 that contains a channel of the one or more channels 108 may be approximately 0.45 to about 1.50 millimeters. In some embodiments, the one or more channels 108 may not cut into the sensor cover 106, so that each of the one or more channels has the same thickness as the sensor cover 106.

In one or more embodiments, the one or more blocks 206 may be made from a similar material or the same material as the sensor cover 106. For example, in one embodiment, the one or more blocks 206 may be integrally formed with the sensor cover 106 via injection molding so that the one or more blocks 206 are the same material as the sensor cover 106. In another example, the one or more blocks may be plastic, silicon, or any other similar material, and connected to the sensor cover 106 as described above.

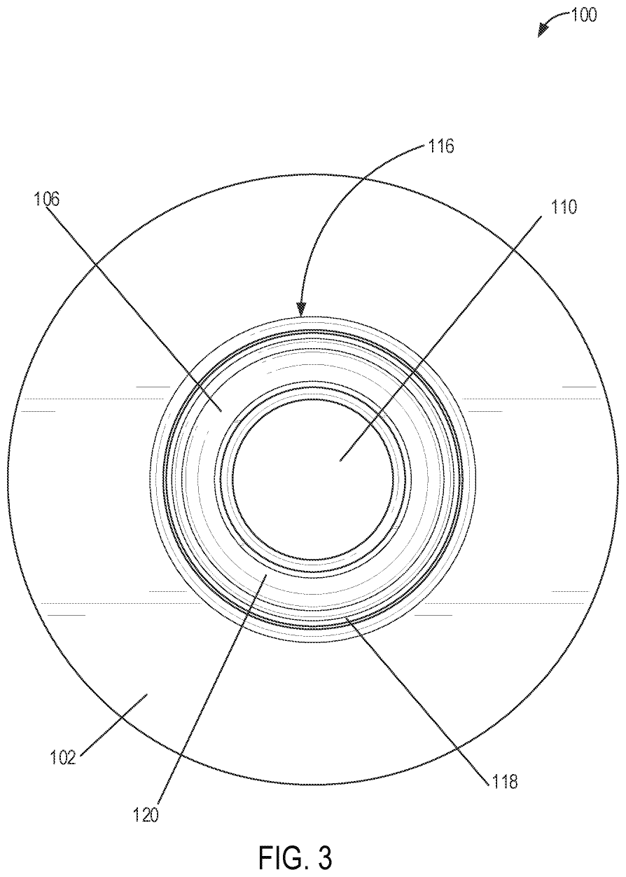

Turning now to FIG. 3, a top view of an exemplary overpatch device 100 is shown, according to one embodiment of the present disclosure. In various embodiments, and as described above, the base 118 and the top area 120 of the sensor cover 106 may be circular, and the diameter of the inner edge 116 of the disk-shaped portion 102 may be larger than the diameter of the opening 110.

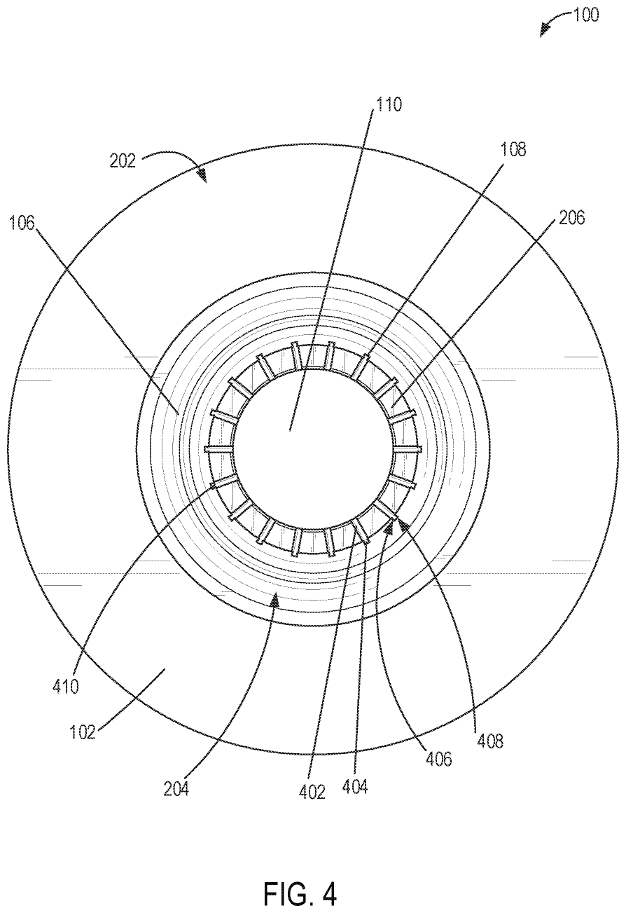

Turning now to FIG. 4, a bottom view of an exemplary overpatch device 100 is shown, according to one embodiment of the present disclosure.

In various embodiments, the sensor cover 106 may include a particular number of one or more blocks 206. In at least one embodiment, the sensor cover 106 may include a suitable number of one or more blocks 206 to secure the biosensor in the overpatch device 100. For example, in one embodiment, the sensor cover 106 may include eighteen blocks in the one or more blocks 206.

In several embodiments, the sensor cover 106 and one or more blocks may define a particular number of one or more channels 108. In some embodiments, the sensor cover 106 may include a suitable number of one or more channels 108 to enable air and moisture to escape from the inside of the sensor cover 106. For example, in many embodiments, the sensor cover 106 may define eighteen channels in the one or more channels. In one embodiment, the ratio of one or more blocks 206 to one or more channels 108 may be 1:1.

In multiple embodiments, the one or more channels 108 may be the shape of the space as defined by the side edges 212 of the adjacent blocks of the one or more blocks 206 that form each channel of the one or more channels 108. In at least one embodiment, the one or more channels 108 may generally be rectangular. In some embodiments, the one or more channels 108 may include a rectangular portion 402 and a trapezoidal-shaped portion 404 extending past the rectangular portion 402. In one or more embodiments, the rectangular portion 402 may have dimensions of approximately 3.0 millimeters in length (e.g., length of the one or more side edges 212), approximately 0.8 millimeters in width (e.g., the distance between two adjacent one or more blocks 206), and approximately 1.0 millimeters in height (e.g., height of the one or more blocks 206). In many embodiments, the length of the rectangular portion 402 may be approximately 0.5 to about 2.5 millimeters, 2.5 to about 3.5 millimeters, and/or 3.5 to about 5.0 millimeters. In several embodiments, the width of the rectangular portion 402 may be approximately 0.1 to about 2.0 millimeters, 2.0 to about 4.0 millimeters, and/or 4.0 to about 5.0 millimeters. In at least one embodiment, the height of the rectangular portion 402 may be approximately 0.1 to about 1.1 millimeters, 1.1 to about 2.0 millimeters, and/or 2.0 to about 5.0 millimeters.

In many embodiments, the trapezoidal portion 404 may include one or more angled sides 406 and a back side 408 that connects the one or more angled sides 406. In one or more embodiments, the interior surface 204 of the sensor cover 106 may form a narrow gap 410 at the connection of the rectangular portion 402 and the trapezoidal portion 404. In at least one embodiment, the narrow gap 410 may be shorter in width than the width of the rectangular portion 402, and may also be smaller in width than the back side 408 of the trapezoidal portion 404. In one embodiment, the angled sides 404 may extend from the narrow gap 410 at equal, opposing angles for a particular distance. In some embodiments, the back side 408 may have a length that is substantially similar to the width of the rectangular portion 402. In another embodiment, instead of the trapezoidal portion 404 extending past the rectangular portion 402, the rectangular portion 402 may extend past the back edge 210 of the one or more blocks 206, such that the portion of the channel that extends past the back edge 210 of the one or more blocks 206 is rectangular.

Turning now to FIG. 5, a side view of an exemplary overpatch device 100 is shown, according to one embodiment of the present disclosure. As shown in FIG. 5 (and as described above) the disk-shaped portion 102 may be connected to the sensor cover 106 by utilizing the adhesive on the bottom surface 202 of the disk-shaped portion 102 to adhere to the exterior surface 112 of the sensor cover 106 at or proximate to the base 118. In at least one embodiment, the disk-shaped portion 102 may connect to the sensor cover 106 so that the base 118 is under the connected portion of the disk-shaped portion 102.

In various embodiments, as shown in FIG. 5, the sensor cover 106 may include a sloped middle area 502 such that the base 118 of the sensor cover 106 is wider than the top area 120 of the sensor cover 106. In some embodiments, the sloped middle area 502 of the sensor cover 106 may generally slope at an angle of approximately 65 degrees. In one or more embodiments, the sloped middle area 502 may slope at an angle of approximately 60 to about 70 degrees. In one embodiment, the sloped middle area 502 may slope at an angle of approximately 70 to about 75 degrees.

In several embodiments, the sensor cover 106 may include curves at the base 118 and the top area 120. In one or more embodiments, the sensor cover 106 curves towards the center of the opening 110 (not shown in FIG. 5) at a certain curvature angle such that the top area 120 is substantially flat at the intersection of the opening 110, with the exterior surface 112 of the sensor cover 106 above the interior surface 204 (not shown in FIG. 5). In some embodiments, the curvature angle at the top area 120 may be approximately 32.5 degrees. In some embodiments, the curvature angle at the top area 120 may be approximately 30 to about 35 degrees. In one embodiment, the curvature angle at the top area 120 may be approximately 35 to about 45 degrees.

In at least one embodiment, the sensor cover 106 curves outwardly, away from the center of the opening 110 (not shown in FIG. 5) at a certain curvature angle such that the base 118 is substantially flat at the outer edge 214 of the base 118, with the exterior surface 112 of the sensor cover 106 above the interior surface 204 (not shown in FIG. 5). In one embodiment, the curvature angle at the base 118 may be approximately 32.5 degrees. In some embodiments, the curvature angle at the base 118 may be approximately 30 to about 35 degrees. In one embodiment, the curvature angle at the base 118 may be approximately 35 to about 45 degrees.

In multiple embodiments, the disk-shaped portion 102 may operatively connect to the sensor cover 106 such that at least a part of the bottom surface 202 of the disk-shaped portion 102 is attached to the exterior surface 112 of the base 118 of the sensor cover 106. In at least one embodiment, the outwardly curved (and substantially flat proximate to the outer edge 214) base 118 of the sensor cover 106 may increase the surface area for the bottom surface 202 of the disk-shaped portion 102 to connect with. In one or more embodiments, the attachment of the bottom surface 202 of the disk-shaped portion 102 to the exterior surface 112 of sensor cover 106 may ensure that the adhesive on the bottom surface 202 of the disk-shaped portion 102 does not touch the sensor inside the sensor cover 106.

In various embodiments, the sensor cover 106 may have a certain height. In at least one embodiment, the height of the sensor cover 106 may be defined as the distance from the interior surface 204 of the substantially flat portion of the base 118 (e.g., the portion of the base 118 that contacts a user when in use) to the exterior surface 112 of the substantially flat portion of the top area 120. In many embodiments, the height of the sensor cover 106 may be any suitable height to contain a sensor within the sensor cover 106. In some embodiments, the height of the sensor cover 106 may be approximately 8.0 millimeters. In at least one embodiment, the height of the sensor cover 106 may be approximately 7.75 to about 8.25 millimeters. In one or more embodiments, the height of the sensor cover 106 may be approximately 8.0 to about 10.0 millimeters. In one embodiment, the height of the sensor cover 106 may be approximately 6.0 to about 8.0 millimeters. In several embodiments, the height of the sensor cover 106 may be approximately 10.0 to about 15.0 millimeters.

Turning now to FIG. 6, an exploded view of an exemplary overpatch device 600 is shown, according to one embodiment of the present disclosure. In various embodiments, the overpatch device 600 may include a sensor 602, a sensor cover 106, a disk-shaped portion 102, and adhesive covers 604 and 606.

In multiple embodiments, the disk-shaped portion 102 may define the circular opening 614 that has inner edge 116. In many embodiments, the inner edge 116 may include the second diameter that is smaller than the first diameter (defined by the outer edge 114). In some embodiments, the second diameter based on the circumference of the inner edge 116 may be any suitable diameter that is less than the first diameter defined by the outer edge 114. In one or more embodiments, the second diameter based on the circumference of the inner edge 116 may be approximately 34 millimeters. In one embodiment, the second diameter may be approximately 30-40 millimeters. In several embodiments, the second diameter may be approximately 25.20 to about 35.30 millimeters.

In many embodiments, the sensor 602 may be any biosensor or other sensor for measuring biomarkers in a human. In one or more embodiments, the sensor cover 106 and disk-shaped portion 102 may be sized so that the sensor 602 may fit securely within the sensor cover 106.

In at least one embodiment, each of the adhesive covers 604 and 606 may include a top surface 610 and a bottom surface 612. In several embodiments, the top surface 610 of each of the adhesive covers 604 and 606 may adhere to the bottom surface 202 of the disk-shaped portion 102 so that the adhesive on the bottom surface 202 does not adhere to an object prior to removing the adhesive covers 604 and 606 from the bottom surface 202 of the disk-shaped portion 102. In some embodiments, the adhesive covers 604 and 606 may be made from a material that may allow a user to easily remove the adhesive covers 604 and 606 from the bottom surface 202 of the disk-shaped portion 102. For example, in one embodiment, the adhesive covers 604 and 606 may be made of a coated paper (e.g., paper with a film coated on it), such that the top surface 610 of each of the adhesive covers 604 and 606 does not distress the adhesive on the bottom surface 202 of the disk-shaped portion 102 upon removal of the adhesive covers 604 and 606 from the disk-shaped portion 102. In one or more embodiments, the adhesive covers 604 and 606 may each include a tab 608 that extends from the top surface 610 and bottom surface 612 of the adhesive covers 604 and 606 so that a user may more easily remove the adhesive covers 604 and 606 from the disk-shaped portion 102. In multiple embodiments, each tab 608 may be any suitable shape that enables a user to more easily remove the adhesive covers 604 and 606 from the disk-shaped portion. In some embodiments, each tab 608 may have a substantially quadrilateral shape with rounded corners.

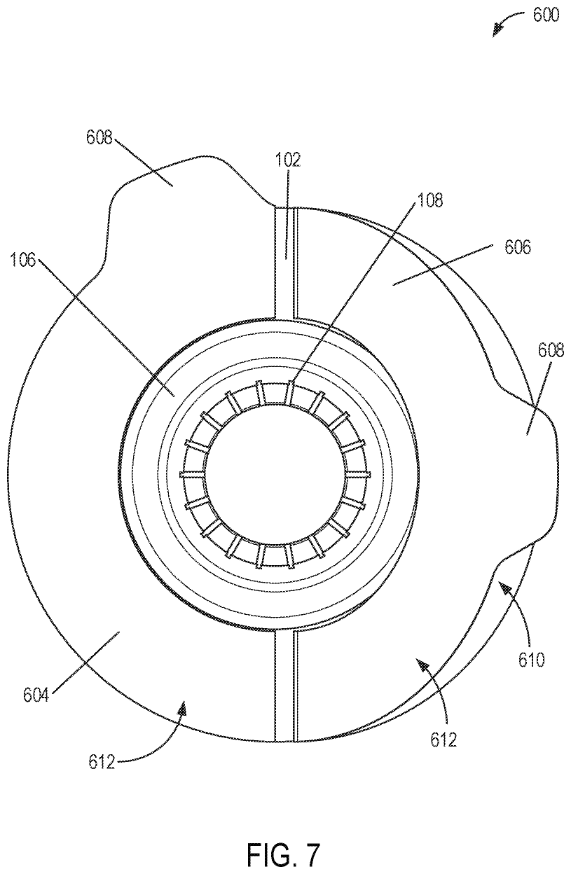

In multiple embodiments, the adhesive cover 604 may be a half-disk shape having a similar width as the disk-shaped portion 102, such that the adhesive cover 604 substantially covers about half of the surface area of the bottom surface 202 of disk-shaped portion 102 (as shown in FIG. 7). In one or more embodiments, the width of the adhesive cover 604 may be approximately 15.73 millimeters. In some embodiments, the width of the adhesive cover 604 may be approximately 12.5 millimeters, approximately 10.0 to about 15.0 millimeters, approximately 20.0 millimeters, and approximately 15.0 to about 25 millimeters. In at least one embodiment, the tab 608 on adhesive cover 604 may project out from the half-circle body of adhesive cover 604. In many embodiments, the tab 608 may extend from any suitable portion of the adhesive cover 604. In one embodiment, the tab 608 may be located proximate to one end of the half-circle body.

In various embodiments, the adhesive cover 604 may include an outer perimeter 616 that in part defines the half-circle body of the adhesive cover 604. In some embodiments, the outer perimeter 616 may have a certain circumference. In one or more embodiments, the tab 608 extends out from adhesive cover 604 such that the outer perimeter 616 diverges from the circumference of the adhesive cover 604 at two points. In at least one embodiment, the tab 608 on adhesive cover 604 may have a length that is defined as a portion of the circumference between the two points the outer perimeter 616 diverges from (e.g., the two points that extend out further than the radius of the circle that the circular portion of the outer perimeter 616 defines). In one embodiment, the tab 608 includes a width that may be the distance the tab 608 extends out from the adhesive cover 604.

In several embodiments, the adhesive cover 606 may be a half-circle shape (e.g., identical or nearly identical to adhesive cover 604), or may be a whole circle that is bent in half, having a similar width as the disk-shaped portion 102. In one or more embodiments, the width of the adhesive cover 606 may be approximately 15.73 millimeters. In at least one embodiment, the width of the adhesive cover 606 may be approximately 12.5 millimeters, approximately 10.0 to about 15.0 millimeters, approximately 20.0 millimeters, and approximately 15.0 to about 25 millimeters. In some embodiments, the adhesive cover 606 substantially covers about half of the surface area of the bottom surface 202 of the disk-shaped portion 102 (as shown in FIG. 7). In one embodiment, if the adhesive cover 606 is a whole circle bent in half, then if the adhesive cover 606 was straightened out, the adhesive cover 606 would cover substantially the entire disk-shaped portion 102. In many embodiments, the tab 608 may extend from any suitable portion of the adhesive cover 606. In at least one embodiment, the tab 608 on adhesive cover 606 may project out from the body of adhesive cover 606 on the portion of the body that is not connected to the disk-shaped portion 102.

In various embodiments, the adhesive cover 606 may include an outer perimeter 618 that in part defines the circular body of the adhesive cover 606. In some embodiments, the outer perimeter 618 may have a certain circumference. In one or more embodiments, the tab 608 extends out from adhesive cover 606 such that the outer perimeter 618 diverges from the circumference of the adhesive cover 606 at two points. In at least one embodiment, the tab 608 on adhesive cover 606 may have a length that is defined as a portion of the circumference between the two points the outer perimeter 618 diverges from (e.g., the two points that extend out further than the radius of the circle that the circular portion of the outer perimeter 618 defines). In one embodiment, the tab 608 includes a width that may be the distance the tab 608 extends out from the adhesive cover 606.

In several embodiments, each tab 608 may have general dimensions of approximately 28.9 millimeters in length by approximately 10.70 millimeters in width. In many embodiments, each tab 608 may have a length of approximately 27.0 to about 30.0 millimeters. In at least one embodiment, each tab 608 may have a length of approximately 26.0 millimeters, approximately 24.0 to about 27.5 millimeters, approximately 29.0 to about 33.0 millimeters, and approximately 31.5 millimeters. In one or more embodiments, each tab 608 may have a width of approximately 9.0 to about 12.0 millimeters. In some embodiments, each tab 608 may have a width of approximately 6.0 to about 9.0 millimeters, approximately 7.5 millimeters, approximately 12.0 to about 15.0 millimeters, and approximately 13.5 millimeters.

Turning now to FIG. 7, a bottom view of an exemplary overpatch device 600 is shown, according to one embodiment of the present disclosure. In multiple embodiments, the overpatch device 700 includes the disk-shaped portion 102, the sensor cover 106, the one or more channels 108, and the adhesive covers 604 and 606 operatively connected to the disk-shaped portion 102 (sensor 602 not shown). In at least one embodiment, the adhesive covers 604 and 606 may operate such that a user may pull each tab 608 on each of the adhesive covers 604 and 606 to remove the adhesive covers 604 and 606, allowing the user to place the disk-shaped portion 102 on the user (or another user) without having to touch the bottom surface 202 of the disk-shaped portion 102.

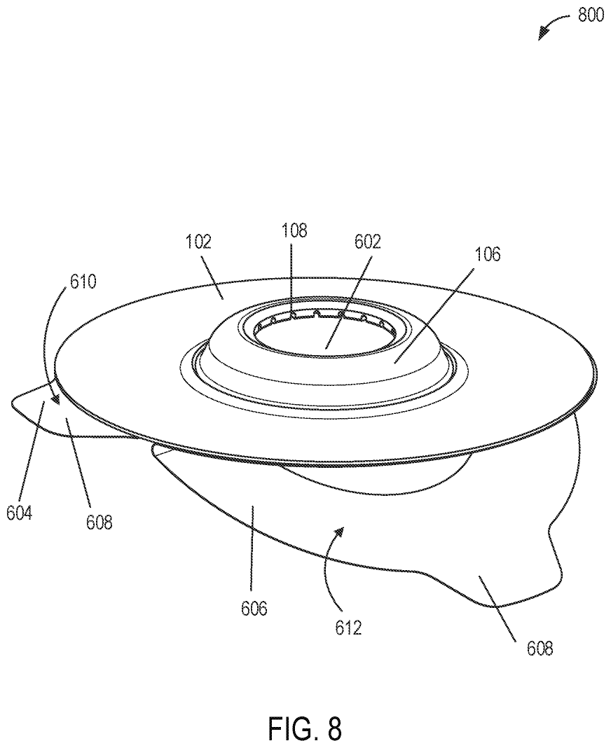

Turning now to FIG. 8, a perspective top view of an exemplary overpatch device 800 is shown, according to one embodiment of the present disclosure. In various embodiments, the overpatch device 800 includes the disk-shaped portion 102, the sensor cover 106, the one or more channels 108, and the adhesive covers 604 and 606 operatively connected to the disk-shaped portion 102, and sensor 602. In one or more embodiments, half of the adhesive cover 606 is connected to the bottom surface 202 of the disk-shaped portion 102, and the other half of adhesive cover 606 bent back so that it mirrors the half connected to the disk-shaped portion 102. In one embodiment, the user may grip and pull the tab on adhesive cover 606 to remove the adhesive cover 606 from the disk-shaped portion 102.

In multiple embodiments, the sensor cover 106 may be placed over the sensor 602, such that the sensor 602 is proximate to the interior surface 204 of the sensor cover 106 (e.g., the sensor is contained by the interior surface 204 for the sensor cover 106 and the user's body. In at least one embodiment, when placing the overpatch device 800 onto a user, the one or more blocks 206 may operate in securing the sensor 602 within the overpatch device 800, because the thickness of the one or more blocks 206 may provide the sensor 602 with less room inside the overpatch device 800 (and the one or more blocks 206 may compress and/or compress the sensor 602 against the skin of a user). In one or more embodiments, depending on the size of the sensor 602, the one or more blocks 206 may be designed different thicknesses to accommodate different sized sensors.

While various aspects have been described in the context of a preferred embodiment, additional aspects, features, and methodologies of the claimed inventions will be readily discernible from the description herein, by those of ordinary skill in the art. Many embodiments and adaptations of the disclosure and claimed inventions other than those herein described, as well as many variations, modifications, and equivalent arrangements and methodologies, will be apparent from or reasonably suggested by the disclosure and the foregoing description thereof, without departing from the substance or scope of the claims. Furthermore, any sequence(s) and/or temporal order of steps of various processes described and claimed herein are those considered to be the best mode contemplated for carrying out the claimed inventions. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed inventions. In addition, some steps may be carried out simultaneously, contemporaneously, or in synchronization with other steps.

The embodiments were chosen and described in order to explain the principles of the claimed inventions and their practical application so as to enable others skilled in the art to utilize the inventions and various embodiments and with various modifications as are suited to the particular use contemplated. Alternative embodiments will become apparent to those skilled in the art to which the claimed inventions pertain without departing from their spirit and scope. Accordingly, the scope of the claimed inventions is defined by the appended claims rather than the foregoing description and the exemplary embodiments described therein.

We claim:

1. An apparatus for securing a biosensor to a human comprising:
a sensor cover for receiving a biosensor device, the cover comprising:
a generally conical-shaped body defining a top opening and a bottom opening and comprising:
an exterior surface sloping from the top opening to the bottom opening;
an interior surface between the top opening and bottom opening comprising one or more protruding portions around a perimeter of the top opening;
one or more channels extending from the interior surface into the body; and
a rim extending outwardly from the exterior surface comprising an upper surface substantially perpendicular to a portion of the sloping exterior surface;
a disk defining a disk opening and comprising a bottom surface; and
an adhesive applied to the disk bottom surface for securing the disk to the sensor cover.

2. The apparatus of claim 1, wherein the one or more protruding portions define a least a portion of the one or more channels.

3. The apparatus of claim 2, wherein the one or more channels extend from the perimeter of the top opening to a first point on the interior surface.

4. The apparatus of claim 3, wherein the one or more protruding portions extend from the perimeter of the top opening to a second point on the interior surface.

5. The apparatus of claim 4, wherein the first point is further from the perimeter of the top opening then the second point.

6. The apparatus of claim 4, wherein each of the one or more channels extend from the perimeter of the top opening approximately 3 millimeters to the first point.

7. The apparatus of claim 6, wherein the disk opening comprises a diameter greater than a diameter of the body bottom opening.

8. The apparatus of claim 7, wherein the disk is secured to the sensor cover by the adhesive attaching to the rim of sensor cover.

9. The apparatus of claim 8, wherein the disk and sensor cover are secured to a human via a portion of the adhesive not attached to the rim of the sensor cover.

10. The apparatus of claim 9, wherein the apparatus further comprises one or more adhesive covers removably attached to the adhesive to protect the adhesive prior to securing the disk to the sensor cover.

11. An apparatus for securing a biosensor to a human comprising:
a sensor cover for receiving a biosensor device, the cover comprising:
a generally conical-shaped body defining a top opening and a bottom opening and comprising:
an exterior surface sloping from the top opening to the bottom opening;
an interior surface between the top opening and bottom opening;
one or more channels extending from the interior surface into the body; and
a rim extending outwardly from the exterior surface comprising an upper surface substantially perpendicular to a portion of the sloping exterior surface;
a disk defining a disk opening and comprising a bottom surface; and
an adhesive applied to the disk bottom surface for securing the disk to the sensor cover.

12. The apparatus of claim 11, wherein the interior surface further comprises one or more protruding portions around a perimeter of the top opening.

13. The apparatus of claim 12, wherein the one or more protruding portions define a least a portion of the one or more channels.

14. The apparatus of claim 13, wherein the one or more channels extend from the perimeter of the top opening to a first point on the interior surface.

15. The apparatus of claim 14, wherein the one or more protruding portions extend from the perimeter of the top opening to a second point on the interior surface.

16. The apparatus of claim 15, wherein the first point is further from the perimeter of the top opening then the second point.

17. The apparatus of claim 15, wherein each of the one or more channels extend from the perimeter of the top opening approximately 3 millimeters to the first point.

18. The apparatus of claim 16, wherein the disk opening comprises a diameter greater than a diameter of the body bottom opening.

19. The apparatus of claim 18, wherein the disk is secured to the sensor cover by the adhesive attaching to the rim of sensor cover.

20. The apparatus of claim 19, wherein the disk and sensor cover are secured to a human via a portion of the adhesive not attached to the rim of the sensor cover.

21. The apparatus of claim 20, wherein the apparatus further comprises one or more adhesive covers removably attached to the adhesive to protect the adhesive prior to securing the disk to the sensor cover.
